(19) United States
(12) Patent Application Publication
(10) Pub. No.: US 2014/0127329 A1
(43) Pub. Date: May 8, 2014

(54) USE OF NITRITES FOR THE TREATMENT OF CEREBRAL AMYLOID ANGIOPATHY, AGE ASSOCIATED DEMENTIA, AND COGNITIVE DECLINE

(71) Applicants: Anthony Giordano, Chesterland, OH (US); Frank Greenway, Baton Rouge, LA (US); Jeffrey Keller, Baton Rouge, LA (US); Christpher Kevil, Shreveport, LA (US)

(72) Inventors: Anthony Giordano, Chesterland, OH (US); Frank Greenway, Baton Rouge, LA (US); Jeffrey Keller, Baton Rouge, LA (US); Christpher Kevil, Shreveport, LA (US)

(73) Assignees: Board of Supervisors of Louisiana State University and Agricultural and Mechanical College, Baton Rouge, LA (US); Thera Vasc Inc., Cleveland, OH (US)

(21) Appl. No.: 14/008,267

(22) PCT Filed: Mar. 30, 2012
(86) PCT No.: PCT/US2012/031459

§ 371 Date: Jan. 8, 2014

Related U.S. Application Data
(60) Provisional application No. 61/470,004, filed on Mar. 31, 2011.

(51) Int. Cl. A61K 33/00; A61K 45/06

(52) U.S. Cl. CPC A61K 33/00; A61K 45/06
USPC 424/718

(57) ABSTRACT

The present invention relates to the medical use of nitrites, such as inorganic nitrites, or any pharmaceutically acceptable salts, solvates, compositions, or prodrugs thereof, in the treatment of conditions that benefit from increased cerebral vascular flow. The pharmaceutical compositions used in these methods, which can be formulated for oral administration, can provide immediate release or extended release of the nitrite ion (NO₂⁻).

### CROSS-REFERENCE TO RELATED APPLICATIONS

This application claims benefit of U.S. Provisional Application No. 61/470,004, filed Mar. 31, 2011, which is hereby incorporated by reference in its entirety.

### BACKGROUND OF THE INVENTION

The present invention relates to pharmaceutical compositions of nitrites and the medical use of these compositions.

Nitrite has been shown to be cytoprotective and have vasodilatory effects and promoting each of these effects in the cerebrovascular architecture can be beneficial in the treatment of diseases and disorders such as cerebral amyloid angiopathy, age associated dementia, and cognitive decline, as well as increasing cerebral blood flow. Increased blood flow and localized nitric oxide production can mediate beneficial effects by slowing the progression of dementia. Nitrite may also be able to reverse the amyloid deposition and other neuropathological features present in the brain of diabetic individuals with dementia. Inorganic nitrite compounds also selectively promote arteriogenesis and angiogenesis in ischemic tissue.

Nitrite therapies that target the vascular system can therefore serve as a useful strategy for the treatment of, e.g., diabetic patients with dementia who have cerebrovascular abnormalities. Present approaches to treating dementia are diffuse and systemic approaches that do not directly address the problem of an insufficient vascular supply or a defective vascular function. Accordingly, methods of inorganic nitrite therapy to restore nitric oxide bioavailability and selectively promote angiogenesis in ischemic tissue can be useful.

### SUMMARY OF THE INVENTION

The present invention relates to pharmaceutical compositions of nitrite (e.g., inorganic nitrite), or a pharmaceutically acceptable salt, solvate, or prodrug thereof, and use of these compositions for the treatment of chronic tissue ischemia, including chronic tissue ischemia associated with a disorder, trauma or a congenital defect.

In a first aspect, the invention features a method for treating or preventing a condition that benefits from increasing cerebral blood flow in a mammal, where the method includes the administration of an effective amount of inorganic nitrite, or a pharmaceutically acceptable salt, solvate, or prodrug thereof.

In some embodiments, the inorganic nitrite, or a pharmaceutically acceptable salt, solvate, or prodrug thereof, is administered as a pharmaceutical composition that further includes a pharmaceutically acceptable excipient.

In some embodiments, administration of the pharmaceutical composition to a human results in a peak plasma concentration of nitrite ion between 0.05 μM-10 μM (e.g., between 0.05 μM-5 μM).

In some embodiments, the peak plasma concentration of nitrite ion is maintained for up to 14 hours. In other embodiments, the peak plasma concentration of nitrite ion is maintained for up to 1 hour.

In some embodiments, the condition is dementia or cognitive decline.

In other embodiments, the dementia is age-associated dementia or vascular dementia

In certain embodiments, the dementia is mild to moderate dementia of the Alzheimer's type or dementia associated with Parkinson's disease.

In further embodiments, the dementia is moderate to severe dementia of the Alzheimer's type or dementia associated with Parkinson's disease.

In still other embodiments, the condition is cerebral amyloid angiopathy.

In some embodiments, the mammal is a human.

In other embodiments, the inorganic nitrite is administered at a dose that is between 0.05 mg-5 mg/kg weight of the human.

In certain embodiments, the pharmaceutical composition is formulated for oral administration.

In other embodiments, the pharmaceutical composition is formulated for extended release.

In still other embodiments, the pharmaceutical composition is formulated for immediate release.

In some embodiments, the pharmaceutical composition is administered concurrently with one or more therapeutic agents for the treatment or prevention of a neurological disorder (e.g., donepezil, rivastigmine, memantine, or galantamine).

In further embodiments, the neurological order is a neurodegenerative disease.

In a second aspect, the invention features a method for increasing cerebral blood flow in a mammal diagnosed with a condition selected from cerebral amyloid angiopathy, age-associated dementia, and cognitive decline, where the method includes the administration of an effective amount of inorganic nitrite, or a pharmaceutically acceptable salt, solvate, or prodrug thereof.

In some embodiments, the inorganic nitrite, or a pharmaceutically acceptable salt, solvate, or prodrug thereof, is administered as a pharmaceutical composition that further includes a pharmaceutically acceptable excipient.

In some embodiments, administration of the pharmaceutical composition to a human results in a peak plasma concentration of nitrite ion between 0.05 μM-10 μM (e.g., between 0.05 μM-5 μM).

In some embodiments, the peak plasma concentration of nitrite ion is maintained for up to 14 hours. In other embodiments, the peak plasma concentration of nitrite ion is maintained for up to 1 hour.

In some embodiments, the condition is dementia or cognitive decline.

In other embodiments, the dementia is age-associated dementia or vascular dementia

In certain embodiments, the dementia is mild to moderate dementia of the Alzheimer's type or dementia associated with Parkinson's disease.

In further embodiments, the dementia is moderate to severe dementia of the Alzheimer's type or dementia associated with Parkinson's disease.

In still other embodiments, the condition is cerebral amyloid angiopathy.

In some embodiments, the mammal is a human.

In other embodiments, the inorganic nitrite is administered at a dose that is between 0.05 mg-5 mg/kg weight of the human.

In certain embodiments, the pharmaceutical composition is formulated for oral administration.

In other embodiments, the pharmaceutical composition is formulated for extended release.

In still other embodiments, the pharmaceutical composition is formulated for immediate release.

In some embodiments, the pharmaceutical composition is administered concurrently with one or more therapeutic agents for the treatment or prevention of a neurological disorder (e.g., donepezil, rivastigmine, memantine, or galantamine).

In further embodiments, the neurological order is a neurodegenerative disease.

As used herein, the term “delayed release” refers to a pharmaceutical preparation, e.g., an orally administered formulation, which passes through the stomach substantially intact and dissolves in the small and/or large intestine (e.g., the colon). In some embodiments, delayed release of the active agent (e.g., nitrite as described herein) results from the use of an enteric coating of an oral medication (e.g., an oral dosage form).

The term an “effective amount” of an agent, as used herein, is that amount sufficient to effect beneficial or desired results, such as clinical results, and, as such, an “effective amount” depends upon the context in which it is being applied.

The terms “extended release” or “sustained release” interchangeably refer to a drug formulation that provides for gradual release of a drug over an extended period of time, e.g., 6-12 hours or more, compared to an immediate release formulation of the same drug. Preferably, although not necessarily, results in substantially constant blood levels of a drug over an extended time period that are within therapeutic levels and fall within a peak plasma concentration range that is between, for example, 0.05-10 μM, 0.1-10 μM, 0.1-5.0 μM, or 0.1-1 μM.

As used herein, the terms “formulated for enteric release” and “enteric formulation” refer to pharmaceutical compositions, e.g., oral dosage forms, for oral administration able to provide protection from dissolution in the high acid (low pH) environment of the stomach. Enteric formulations can be obtained by, for example, incorporating into the pharmaceutical composition a polymer resistant to dissolution in gastric juices. In some embodiments, the polymers have an optimum pH for dissolution in the range of approx. 5.0 to 7.0 (“pH sensitive polymers”). Exemplary polymers include methacrylate acid copolymers that are known by the trade name Eudragit® (e.g., Eudragit® L100, Eudragit® S100, Eudragit® L-30D, Eudragit® FS 30D, and Eudragit® L100-55), cellulose acetate phthalate, cellulose acetate trimellitiate, polyvinyl acetate phthalate (e.g., Coateric®), hydroxyethylcellulose phthalate, hydroxypropyl methylcellulose phthalate, or shellac, or an aqueous dispersion thereof. Aqueous dispersions of these polymers include dispersions of cellulose acetate phthalate (Aquateric®) or shellac (e.g., MarCoat 125 and 125N). An enteric formulation reduces the percentage of the administered dose released into the stomach by at least 50%, 60%, 70%, 80%, 90%, 95%, or even 98% in comparison to an immediate release formulation. Where such a polymer coats a tablet or capsule, this coat is also referred to as an “enteric coating.”

By “immediate release” is meant that the agent (e.g., nitrite or nitrate ion), as formulated in a unit dosage form, has a dissolution release profile under in vitro conditions in which at least 55%, 65%, 75%, 85%, or 95% of the agent is released within the first two hours of administration to, e.g., a human. Desirably, the agent formulated in a unit dosage has a dissolution release profile under in vitro conditions in which at least 50%, 65%, 75%, 85%, 90%, or 95% of the agent is released within the first 30 minutes, 45 minutes, or 60 minutes of administration.

The term “pharmaceutical composition,” as used herein, represents a composition containing a compound described herein (e.g., inorganic nitrite, or any pharmaceutically acceptable salt, solvate, or prodrug thereof), formulated with a pharmaceutically acceptable excipient, and typically manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other formulation described herein.

A “pharmaceutically acceptable excipient,” as used herein, refers any ingredient other than the compounds described herein (for example, a vehicle capable of suspending or dissolving the active compound) and having the properties of being nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, or waters of hydration. Exemplary excipients include, but are not limited to: butylated hydroxytoluene (BHT), calcium carbonate, calcium phosphate (dibasic), calcium stearate, croscarmellose, cross-linked polyvinyl pyrrolidone, citric acid, crospovidone, cysteine, ethylcellulose, gelatin, hydroxypropyl cellulose, hydroxypropyl methylcellulose, lactose, magnesium stearate, maltitol, maltose, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol.

The term “pharmaceutically acceptable prodrugs” as used herein, represents those prodrugs of the compounds of the present invention which are, within the scope of sound medical judgment, suitable for use in contact with the tissues of humans and animals with undue toxicity, irritation, allergic response, and the like, commensurate with a reasonable benefit/risk ratio, and effective for their intended use, as well as the zwitterionic forms, where possible, of the compounds of the invention.

The term “pharmaceutically acceptable salt,” as use herein, represents those salts which are, within the scope of sound medical judgment, suitable for use in contact with the tissues of humans and animals without undue toxicity, irritation, allergic response and the like and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds of the invention or separately by reacting the free base group with a suitable organic or inorganic acid. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

The terms “pharmaceutically acceptable solvate” or “solvate,” as used herein, means a compound of the invention wherein molecules of a suitable solvent are incorporated in the crystal lattice. A suitable solvent is physiologically tolerable at the administered dose. For example, solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.”

The term “prevent,” as used herein, refers to prophylactic treatment or treatment that prevents one or more symptoms or conditions of a disease, disorder, or conditions described herein (e.g., chronic tissue ischemia). Treatment can be initiated, for example, prior to (“pre-exposure prophylaxis”) or following (“post-exposure prophylaxis”) an event that precedes the onset of the disease, disorder, or conditions. Treatment that includes administration of a compound of the invention, or a pharmaceutical composition thereof, can be acute, short-term, or chronic. The doses administered may be varied during the course of preventive treatment.

The term “prodrug,” as used herein, represents compounds which are rapidly transformed in vivo to the parent compound of the above formula. Prodrugs also encompass bioequivalent compounds that, when administered to a human, lead to the in vivo formation of nitrite ion (NO₂⁻) or nitrous oxide (NO). A thorough discussion is provided in T. Higuchi and V. Stella, Pro-drugs as Novel Delivery Systems, Vol. 14 of the A.C.S. Symposium Series, and Edward B. Roche, ed., Bioreversible Carriers in Drug Design, American Pharmaceutical Association and Pergamon Press, 1987, each of which is incorporated herein by reference. Preferably, prodrugs of the compounds of the present invention are pharmaceutically acceptable such as those described in EP 1336602A1, which is herein incorporated by reference.

As used herein, and as well understood in the art, “treatment” is an approach for obtaining beneficial or desired results, such as clinical results. Beneficial or desired results can include, but are not limited to, alleviation or amelioration of one or more symptoms or conditions; diminishment of extent of disease, disorder, or condition; stabilized (i.e. not worsening) state of disease, disorder, or condition; preventing spread of disease, disorder, or condition; delay or slowing the progress of the disease, disorder, or condition; amelioration or palliation of the disease, disorder, or condition; and remission (whether partial or total), whether detectable or undetectable. “Treatment” can also mean prolonging survival as compared to expected survival if not receiving treatment. As used herein, the terms “treating” and “treatment” can also refer to delaying the onset of, impeding or reversing the progress of, or alleviating either the disease or condition to which the term applies, or one or more symptoms of such disease or condition.

The term “unit dosage forms” refers to physically discrete units suitable as unitary dosages for human subjects and other mammals, each unit containing a predetermined quantity of active material calculated to produce the desired therapeutic effect, in association with any suitable pharmaceutical excipient or excipients.

As used herein, the term “plasma concentration” refers to the amount of nitrite ion present in the plasma of a treated subject (e.g., as measured in a rabbit using an assay described below or in a human).

Other features and advantages of the invention will be apparent from the following Detailed Description and the claims.

### DETAILED DESCRIPTION

The invention features the use of nitrite (e.g., inorganic nitrite), or a pharmaceutically acceptable salt, solvate, or prodrug thereof, in therapeutic methods to increase cerebral vascular blood flow in a patient in need thereof. These methods can be useful for the treatment of e.g., cerebral amyloid angiopathy (CAA), as well as age associated dementia and cognitive decline.

### Nitrite

Inorganic Nitrite

The pharmaceutically acceptable compositions of the invention include inorganic nitrite, e.g., a salt or ester of nitrous acid (HNO₂), or a pharmaceutically acceptable salt thereof. Nitrite salts can include, without limitation, salts of alkali metals, e.g., sodium, potassium; salts of alkaline earth metals, e.g., calcium, magnesium, and barium; and salts of organic bases, e.g., amine bases and inorganic bases. Compounds of the invention also include all isotopes of atoms occurring in the intermediate or final compounds. Isotopes include those atoms having the same atomic number but different mass numbers. For example, isotopes of hydrogen include tritium and deuterium. The term “compound,” as used herein with respect to any inorganic nitrite or pharmaceutically acceptable salt, solvate, or prodrug thereof. All compounds, and pharmaceutical acceptable salts thereof; are also meant to include solvated (e.g., hydrated) forms. Nitrite has the chemical formula NO₂⁻ and may exist as an ion in water. Sodium nitrite has the chemical formula NaNO₂ and typically dissolves in water to form the sodium ion Na⁺ and the nitrite ion NO₂⁻. It will further be understood that the present invention encompasses all such solvated forms (e.g., hydrates) of the nitrite compounds. Exemplary nitrite compounds are described in WO 2008/105730, which is hereby incorporated by reference.

In addition to sodium nitrite, representative inorganic nitrite compounds include: ammonium nitrite (NH₄NO₂), barium nitrite (Ba(NO₂)₂; e.g., anhydrous barium nitrite or barium nitrite monohydrate), calcium nitrite (Ca(NO₂)₂; e.g., anhydrous calcium nitrite or calcium nitrite monohydrate), cesium nitrite (CsNO₂), cobalt(II) nitrite (Co(NO₂)₂), cobalt(III) potassium nitrite (CoK₃(NO₂)₆; e.g., cobalt(III) potassium nitrite sesquihydrate), lithium nitrite (LiNO₂; e.g., anhydrous lithium nitrite or lithium nitrite monohydrate), magnesium nitrite (MgNO₂; e.g., magnesium nitrite trihydrate), postassium nitrite (KNO₂), rubidium nitrite (RbNO₂), silver(I) nitrite (AgNO₂), strontium nitrite (Sr(NO₂)₂), and zinc nitrite (Zn(NO₂)₂).

The compounds of the present invention can be prepared in a variety of ways known to one of ordinary skill in the art of chemical synthesis. Methods for preparing nitrite salts are well known in the art and a wide range of precursors and nitrite salts are readily available commercially. Nitrites of the alkali and alkaline earth metals can be synthesized by reacting a mixture of nitrogen monoxide (NO) and nitrogen dioxide (NO₂) with a corresponding metal hydroxide solution, as well as through the thermal decomposition of the corresponding nitrate. Other nitrites are available through the reduction of the corresponding nitrates.

The present compounds can be prepared from readily available starting materials using the methods and procedures known in the art. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one of ordinary skill in the art by routine optimization procedures.

Suitable pharmaceutically acceptable salts include, for example, sodium nitrite, potassium nitrite, or calcium nitrite. Still other exemplary salts are found in Remington's Pharmaceutical Sciences, 17th ed., Mack Publishing Company, Easton, Pa., 1985, p. 1418, Berge et al., J. Pharmaceutical Sciences 66:1-19, 1977 and Pharmaceutical Salts: Properties, Selection, and Use, (Eds. P. H. Stahl and C. G. Wermuth), Wiley-VCH, 2008, each of which is incorporated herein by reference in its entirety.

### Pharmaceutical Compositions

The methods described herein can also include the administrations of pharmaceutically acceptable compositions that include inorganic nitrite, e.g., a salt of nitrous acid (HNO₂) such as NaNO₂, or a pharmaceutically acceptable salt, solvate, or prodrug thereof (e.g., nitrates). When employed as pharmaceuticals, any of the present compounds can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical, parenteral, intravenous, intra-arterial, subcutaneous, intramuscular, intracranial, intraorbital, ophthalmic, intraventricular, intracapsular, intraspinal, intracisternal, intraperitoneal, intranasal, aerosol, by suppositories, or oral administration.

This invention also includes pharmaceutical compositions which can contain one or more pharmaceutically acceptable carriers. In making the pharmaceutical compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semisolid, or liquid material (e.g., normal saline), which acts as a vehicle, carrier or medium for the active ingredient. Thus, the compositions can be in the form of tablets, powders, lozenges, sachets, cachets, elixirs, suspensions, emulsions, solutions, syrups, and soft and hard gelatin capsules. As is known in the art, the type of diluent can vary depending upon the intended route of administration. The resulting compositions can include additional agents, such as preservatives.

The therapeutic agents of the invention can be administered alone, or in a mixture, in the presence of a pharmaceutically acceptable excipient or carrier. The excipient or carrier is selected on the basis of the mode and route of administration. Suitable pharmaceutical carriers, as well as pharmaceutical necessities for use in pharmaceutical formulations, are described in Remington: The Science and Practice of Pharmacy, 21^{st} Ed., Gennaro, Ed., Lippencott Williams & Wilkins (2005), a well-known reference text in this field, and in the USP/NF (United States Pharmacopeia and the National Formulary). In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g. about 40 mesh.

Examples of suitable excipients are lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. Other exemplary excipients are described in Handbook of Pharmaceutical Excipients, 6^{th} Edition, Rowe et al., Eds., Pharmaceutical Press (2009).

The methods described herein can include the admnitration of nitrate salts, or prodrugs or pharmaceutical compositions thereof, or other therapeutic agents. Exemplary nitrate salts are described in WO 2008/105730. Exemplary therapeutic agents that may be included in the compositions described herein are cardiovascular therapeutics (e.g., anti-thrombotics (e.g. dipyridamole, clopidogrel, and the like), anti-hypertensives (e.g., Ca⁺⁺ channel blockers, AT-2 blockers, ACE inhibitors, and the like), anti-cholesterols (e.g., statins, fibrates, and the like), and thiazolidinedione therapeutics.

The pharmaceutical compositions can be formulated so as to provide immediate, extended, or delayed release of the active ingredient after administration to the patient by employing procedures known in the art.

The compositions can be formulated in a unit dosage form, each dosage containing, e.g., 0.1-500 mg of the active ingredient. For example, the dosages can contain from about 0.1 mg to about 50 mg, from about 0.1 mg to about 40 mg, from about 0.1 mg to about 20 mg, from about 0.1 mg to about 10 mg, from about 0.2 mg to about 20 mg, from about 0.3 mg to about 15 mg, from about 0.4 mg to about 10 mg, from about 0.5 mg to about 1 mg; from about 0.5 mg to about 100 mg, from about 0.5 mg to about 50 mg, from about 0.5 mg to about 30 mg, from about 0.5 mg to about 20 mg, from about 0.5 mg to about 10 mg, from about 0.5 mg to about 5 mg; from about 1 mg from to about 50 mg, from about 1 mg to about 30 mg, from about 1 mg to about 20 mg, from about 1 mg to about 10 mg, from about 1 mg to about 5 mg; from about 5 mg to about 50 mg, from about 5 mg to about 20 mg, from about 5 mg to about 10 mg; from about 10 mg to about 100 mg, from about 20 mg to about 200 mg, from about 30 mg to about 150 mg, from about 40 mg to about 100 mg, from about 50 mg to about 100 mg of the active ingredient, from about 50 mg to about 300 mg, from about 50 mg to about 250 mg, from about 100 mg to about 300 mg, or, from about 100 mg to about 250 mg of the active ingredient. For preparing solid compositions such as tablets, the principal active ingredient is mixed with one or more pharmaceutical excipients to form a solid bulk formulation composition containing a homogeneous mixture of a compound of the present invention. When referring to these bulk formulation compositions as homogeneous, the active ingredient is typically dispersed evenly throughout the composition so that the composition can be readily subdivided into equally effective unit dosage forms such as tablets and capsules. This solid bulk formulation is then subdivided into unit dosage forms of the type described above containing from, for example, 0.1 to about 500 mg of the active ingredient of the present invention.

Compositions for Oral Administration

The pharmaceutical compositions contemplated by the invention include those formulated for oral administration (“oral dosage forms”). Oral dosage forms can be, for example, in the form of tablets, capsules, a liquid solution or suspension, a powder, or liquid or solid crystals, which contain the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

Formulations for oral administration may also be presented as chewable tablets, as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Controlled release compositions for oral use may be constructed to release the active drug by controlling the dissolution and/or the diffusion of the active drug substance. Any of a number of strategies can be pursued in order to obtain controlled release and the targeted plasma concentration vs time profile. In one example, controlled release is obtained by appropriate selection of various formulation parameters and ingredients, including, e.g., various types of controlled release compositions and coatings. Thus, the drug is formulated with appropriate excipients into a pharmaceutical composition that, upon administration, releases the drug in a controlled manner. Examples include single or multiple unit tablet or capsule compositions, oil solutions, suspensions, emulsions, microcapsules, microspheres, nanoparticles, patches, and liposomes. In certain embodiments, compositions include biodegradable, pH, and/or temperature-sensitive polymer coatings.

Dissolution or diffusion controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

The liquid forms in which the compounds and compositions of the present invention can be incorporated for administration orally include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions suitable for oral mucosal administration (e.g., buccal or sublingual administration) include tablets, lozenges, and pastilles, where the active ingredient is formulated with a carrier, such as sugar, acacia, tragacanth, or gelatin and glycerine.

Coatings

The pharmaceutical compositions formulated for oral delivery, such as tablets or capsules of the present invention can be coated or otherwise compounded to provide a dosage form affording the advantage of delayed or extended release. The coating may be adapted to release the active drug substance in a predetermined pattern (e.g., in order to achieve a controlled release formulation) or it may be adapted not to release the active drug substance until after passage of the stomach, e.g., by use of an enteric coating (e.g., polymers that are pH-sensitive (“pH controlled release”), polymers with a slow or pH-dependent rate of swelling, dissolution or erosion (“time-controlled release”), polymers that are degraded by enzymes (“enzyme-controlled release” or “biodegradable release”) and polymers that form firm layers that are destroyed by an increase in pressure (“pressure-controlled release”). Exemplary enteric coatings that can be used in the pharmaceutical compositions described herein include sugar coatings, film coatings (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), or coatings based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose. Furthermore, a time delay material such as, for example, glyceryl monostearate or glyceryl distearate, may be employed.

For example, the tablet or capsule can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release.

When an enteric coating is used, desirably, a substantial amount of the drug is released in the lower gastrointestinal tract.

In addition to coatings that effect delayed or extended release, the solid tablet compositions may include a coating adapted to protect the composition from unwanted chemical changes (e.g., chemical degradation prior to the release of the active drug substance). The coating may be applied on the solid dosage form in a similar manner as that described in Encyclopedia of Pharmaceutical Technology, vols. 5 and 6, Eds. Swarbrick and Boyland, 2000.

Parenteral Administration

Within the scope of the present invention are also parenteral depot systems from biodegradable polymers. These systems are injected or implanted into the muscle or subcutaneous tissue and release the incorporated drug over extended periods of time, ranging from several days to several months. Both the characteristics of the polymer and the structure of the device can control the release kinetics which can be either continuous or pulsatile. Polymer-based parenteral depot systems can be classified as implants or microparticles. The former are cylindrical devices injected into the subcutaneous tissue whereas the latter are defined as spherical particles in the range of 10-100 μm. Extrusion, compression or injection molding are used to manufacture implants whereas for microparticles, the phase separation method, the spray-drying technique and the water-in-oil-in-water emulsion techniques are frequently employed. The most commonly used biodegradable polymers to form microparticles are polyesters from lactic and/or glycolic acid, e.g. poly(glycolic acid) and poly(L-lactic acid) (PLG/PLA microspheres). Of particular interest are in situ forming depot systems, such as thermoplastic pastes and gelling systems formed by solidification, by cooling, or due to the sol-gel transition, cross-linking systems and organogels formed by amphiphilic lipids. Examples of thermosensitive polymers used in the aforementioned systems include, N-isopropylacrylamide, poloxamers (ethylene oxide and propylene oxide block copolymers, such as poloxamer 188 and 407), poly(N-vinyl caprolactam), poly(siloethylene glycol), polyphosphazenes derivatives and PLGA-PEG-PLGA.

Mucosal Drug Delivery

Mucosal drug delivery (e.g., drug delivery via the mucosal linings of the nasal, rectal, vaginal, ocular, or oral cavities) can also be used in the methods described herein. Methods for oral mucosal drug delivery include sublingual administration (via mucosal membranes lining the floor of the mouth), buccal administration (via mucosal membranes lining the cheeks), and local delivery (Harris et al., Journal of Pharmaceutical Sciences, 81(1): 1-10, 1992)

Oral transmucosal absorption is generally rapid because of the rich vascular supply to the mucosa and allows for a rapid rise in blood concentrations of the therapeutic (“American Academy of Pediatrics: Alternative Routes of Drug Administration—Advantages and Disadvantages (Subject Review),” Pediatrics, 100(1):143-152, 1997).

For buccal administration, the compositions may take the form of, e.g., tablets, lozenges, etc. formulated in a conventional manner. Permeation enhancers can also be used in buccal drug delivery. Exemplary enhancers include 23-lauryl ether, aprotinin, azone, benzalkonium chloride, cetylpyridinium chloride, cetyltrimethylammonium bromide, cyclodextrin, dextran sulfate, lauric acid, lysophosphatidylcholine, methol, methoxysalicylate, methyloleate, oleic acid, phosphatidylcholine, polyoxyethylene, polysorbate 80, sodium EDTA, sodium glycholate, sodium glycodeoxycholate, sodium lauryl sulfate, sodium salicylate, sodium taurocholate, sodium taurodeoxycholate, sulfoxides, and alkyl glycosides. Bioadhesive polymers have extensively been employed in buccal drug delivery systems and include cyanoacrylate, polyacrylic acid, hydroxypropyl methylcellulose, and poly methacrylate polymers, as well as hyaluronic acidand chitosan.

Liquid drug formulations (e.g., suitable for use with nebulizers and liquid spray devices and electrohydrodynamic (EHD) aerosol devices) can also be used. Other methods of formulating liquid drug solutions or suspension suitable for use in aerosol devices are known to those of skill in the art (see, e.g., Biesalski, U.S. Pat. No. 5,112,598, and Biesalski, U.S. Pat. No. 5,556,611).

Formulations for sublingual administration can also be used, including powders and aerosol formulations. Exemplary forumulations include rapidly disintegrating tablets and liquid-filled soft gelatin capsules.

Dosing Regimes

The present methods for treating chronic tissue ischemia are carried out by administering an inorganic nitrite or nitrate for a time and in an amount sufficient to result in the growth of new blood vessels in the ischemic tissue.

The amount and frequency of administration of the compositions can vary depending on, for example, what is being administered, the state of the patient, and the manner of administration. In therapeutic applications, compositions can be administered to a patient suffering from chronic tissue ischemia in an amount sufficient to relieve or least partially relieve the symptoms of chronic tissue ischemia and its complications. The dosage is likely to depend on such variables as the type and extent of progression of the chronic tissue ischemia, the severity of the chronic tissue ischemia, the age, weight and general condition of the particular patient, the relative biological efficacy of the composition selected, formulation of the excipient, the route of administration, and the judgment of the attending clinician. Effective doses can be extrapolated from dose-response curves derived from in vitro or animal model test system. An effective dose is a dose that produces a desirable clinical outcome by, for example, improving a sign or symptom of chronic tissue ischemia or slowing its progression.

The amount of inorganic nitrite per dose can vary. For example, a subject can receive from about 0.1 μg/kg to about 10,000 μg/kg. Generally, the nitrite is administered in an amount such that the peak plasma concentration ranges from 150 nM-250 μM. Exemplary dosage amounts can fall between 0.1-5000 μg/kg, 100-1500 μg/kg, 100-350 μg/kg, 340-750 μg/kg, or 750-1000 μg/kg. Exemplary dosages can 0.25, 0.5, 0.75, 1°, or 2 mg/kg. In another embodiment, the administered dosage can range from 0.05-5 mmol of nitrate (e.g., 0.089-3.9 mmol) or 0.1-50 μmol of nitrite (e.g., 0.1-25 μmol or 0.4-20 μmol).

The plasma concentration of nitrate or nitrite ion can also be measured according to methods known in the art. Exemplary peak plasma concentrations of nitrite can range from 0.05-10 μM, 0.1-10 μM, 0.1-5.0 μM, or 0.1-1 μM. Alternatively, the average plasma levels of nitrate can range from 400-1200 μM (e.g., between 500-1000 μM) or between 50-250 μM (e.g., between 40-200 μM). In some embodiments where sustained release of the drug is desirable, the peak plasma concentrations (e.g., of nitrite or nitrate) may be maintained for 6-14 hours, e.g., for 6-12 or 6-10 hours. In other embodiments where immediate release of the drug is desirable, the peak plasma concentration (e.g., of nitrite or of nitrate) may be maintained for, e.g., 30 minutes.

The frequency of treatment may also vary. The subject can be treated one or more times per day with nitrite or nitrate ion (e.g., once, twice, three, four or more times) or every so-many hours (e.g., about every 2, 4, 6, 8, 12, or 24 hours). Preferably, the pharmaceutical composition is administered 1 or 2 times per 24 hours. The time course of treatment may be of varying duration, e.g., for two, three, four, five, six, seven, eight, nine, ten or more days. For example, the treatment can be twice a day for three days, twice a day for seven days, twice a day for ten days. Treatment cycles can be repeated at intervals, for example weekly, bimonthly or monthly, which are separated by periods in which no treatment is given. The treatment can be a single treatment or can last as long as the life span of the subject (e.g., many years).

### Kits

Any of the pharmaceutical compositions of the invention described herein can be used together with a set of instructions, i.e., to form a kit. The kit may include instructions for use of the pharmaceutical compositions as a therapy as described herein. For example, the instructions may provide dosing and therapeutic regimes for use of the compounds of the invention to reduce chronic tissue ischemia.

### Methods of Treatment: Modulating Cerebral Vascular Blood Flow

Treatment of Diabetic Patients with Dementia

Diabetes mellitus is a significant contributor to cognitive dysfunction and increases the risk of dementia in Alzheimer's disease and in patients with cerebral vascular insufficiency. Both diabetes and Alzheimer's disease are age-related diseases, and, due to the increasing prevalence of these diseases, they have become major public health concerns. Nearly 23.6 million people are reported to suffer from diabetes in US, and the prevalence of the disease is predicted to rapidly increase in the near future. The Center for Disease Control and Prevention recently reported that Alzheimer's disease is the 6th leading cause of mortality in adults in the United States. Approximately 5 million people in the US suffer from Alzheimer's disease, with a projected 13.4 million by the year 2050. Further, studies have shown that 80% of Alzheimer patients have associated type II diabetes.

Alzheimer's disease is closely related to cerebral amyloid angiopathy (CAA) which is associated with cognitive decline as well stroke. CAA is present in 80-85% of patients with Alzheimer's disease and results in β-amyloid deposition in the walls of small to medium sized arteries. Both Alzheimer's disease and CAA are progressive, fatal diseases. These cerebral vascular abnormalities may provide pathological bases for the genesis of both Alzheimer's disease and cerebral amyloid angiopathy.

Numerous epidemiological studies have demonstrated that patients with diabetes have an increased risk of developing Alzheimer's disease compared with non diabetic patients. These findings also suggest that diabetes mellitus affects the fundamental Alzheimer's disease pathogenesis. Both Type I and Type II diabetes mellitus are found to be associated with changes in mental cognition and flexibility. There are various studies demonstrating possible mechanistic pathways linking diabetes and Alzheimer's disease-associated dementia, including: abnormal protein processing, abnormalities in insulin signaling, dysregulated glucose metabolism, oxidative stress, the formation of advanced glycation end products, and the activation of inflammatory pathways.

Further, studies have established that insulin directly affects β-amyloid metabolism and impedes its removal from brain. Moreover, other studies have reported that insulin increased the extracellular β-amyloid level by specifically modulating γ-secretase activity or by increasing its secretion from neurons. Another study reports that β-amyloid breakdown by insulin-degrading enzyme, which is also a major β-amyloid degrading enzyme, is competitively inhibited by insulin, resulting in decreased β-amyloid degradation. Furthermore, an animal study done in a hyperinsulinemic Alzheimer's disease animal model showed a decrease in the brain insulin-degrading enzyme level. Taken together, these studies suggest that insulin treatment of diabetic patients may actually exascerbate Alzheimer's disease pathology.

Vascular lesions also play a significant pathophysiologic role in the development of dementia of Alzheimer's disease. Cerebral amyloid angiopathy is often associated with the presence of ischemic or hemorrhagic lesions in the brains of patients with Alzheimer's disease. Patients with cerebral amyloid angiopathy present with a progressive course due to the additive effects of severe vascular amyloid, cortical hemorrhages, cortical infarctions, white matter destruction, and accumulation of neuritic plaques. Cerebral amyloid angiopathy can lead to intracranial hemorrhage (ICH) and dementia which results in cognitive impairment in diabetic patients. Studies have linked diminished blood flow to the brain to deterioration in cognition. It has also been reported that cerebral hypo-perfusion contributes to clinical dementia suggesting that cerebrovascular abnormalities are a major cause of cognitive decline in demented diabetic patients.

There is no cure available for dementia and the currently available drugs attempt to slow the progression of the disease. Most of the drugs given to improve congnitive function are acetylcholinesterase inhibitors, and the FDA-approved drugs for the treatment of Alzheimer's include donepezil (Aricept), galantamine (Razadyne), memantine (Namenda), and rivastigmine (Exelon). Alternative disease-modifying approaches for treatment of this disease include targeting the production of the β-amyloid peptide in an effort to clear the plaques and modulation of β-amyloid production by acting on particular enzymes that break down the amyloid protein precursor using inhibitors of gamma and beta secretases. These approaches, however, were not found to be efficacious and were associated with safety issues.

Since diabetic patients with dementia have cerebrovascular abnormalities, nitrite therapies targeting the vascular system can prove useful. The basis of inorganic nitrite therapy is to restore nitric oxide bioavailability and selectively promote angiogenesis in ischemic tissue. Nitrite has been shown to be cytoprotective and to have vasodilatory effects; accordingly, promoting each of these effects in the cerebrovascular architecture of the aged diabetic brain may have beneficial therapeutic effects. Increased blood flow and localized nitric oxide production are expected to mediate beneficial effects by impeding the progression of dementia.

Nitrite may also be able to reverse the amyloid deposition and other neuropathological features present in the brain of diabetic individuals with dementia. The present approaches to treating dementia are diffuse, systemic approaches that do not directly address the problem of an insufficient vascular supply or a defective vascular function. The selective angiogenic activity of nitrite can represent a true breakthrough in the field of diabetes related dementia and offers an opportunity to effectively treat the large number of patients with this disorder.

Age-Associated Dementia and Cognitive Decline

According to current data it is believed that one in seven individuals over the age of 65 and 50% of individuals >85 have age-associated dementia. While Alzheimer's disease (AD) is the most common form of dementia in the elderly, other common forms of dementia in the elderly include vascular dementias such as cerebral amyloid angiopathy (CAA). It is well known that there is considerable cross-talk between AD and vascular dementia with the majority of demented elderly individuals having some form of mixed dementia (AD+vascular dementia). Even in the case of AD, there is significant evidence that cerebral vascular abnormalities may provide the pathological basis for the genesis of AD. Currently there are no FDA approved treatments which modify AD or vascular dementia. Therapies designed to ameliorate cerebrovascular abnormalities in the elderly may be beneficial in treating dementia in the elderly due to AD and/or vascular dementia.

Inorganic nitrite therapy has been demonstrated to be a selective nitric oxide donor during hypoxia and acidosis. Typically, nitrite is derived from nitrate rich diet. Dietary nitrate is absorbed from the upper part of the intestine and transported via plasma into the salivary glands where it is ultimately reduced to nitrite by oral symbiotic bacteria. Nitrite is a circulating and tissue storage form of the gaseous nitric oxide molecule, a key regulator of cardiovascular health and therapeutic angiogenesis. Thus, nitrite is an endogenous modulator with the potential to treat cardiovascular diseases due to the selective conversion of sodium nitrite back to nitric oxide only in the ischemic region without any effect on non-ischemic tissues. It is also known that risk factors associated with cardiovascular disease are inversely correlated to circulating plasma levels of nitrite. These studies suggest that circulating nitrite levels are important modulators of endothelial health. Moreover, cognitive decline in dementia patients is associated with poor cerebral perfusion and ischemia, and aging is associated with endothelial nitric oxide synthase dysfunction which results in reduced nitric oxide levels. The nitrite and nitrate therapies described herein can lead to increases in nitric oxide levels and thus improve tissue perfusion independent of nitric oxide synthase in the endothelium.

The beneficial effects of nitrite therapy have been linked to the stimulation of nitric oxide formation, angiogenesis, arteriogenesis, increased blood flow, increased mitochondrial function, decreased oxidative stress, and decreased expression of inflammatory molecules and increased expression of wound healing and cell proliferation molecules at the site of injury. Promoting these events in the cerebrovascular architecture of the aged brain may have beneficial effects towards age-related dementia. Increased blood flow and localized nitric oxide production, both linked to beneficial effects of sodium nitrite in peripheral vascular injury, may mediate beneficial effects in regards to impeding the progression of dementia in the elderly. Such studies may also be able to reverse the development of amyloid deposition or other neuropathological features present in the brain of individuals with age-related dementia.

The present compositions can also be formulated in combination with one or more additional active ingredients, which can include any pharmaceutical agent such as antihypertensives, anti-diabetic agents, statins, anti-platelet agents (clopidogrel, cilostazol, and dipyridamole), antibodies, immune suppressants, anti-inflammatory agents, antibiotics, chemotherapeutics, and the like. In another embodiment, one or more pharmaceutical agents can be administered in combination with the nitrite formulations described herein. Exemplary pharmaceutical agents that can be administered in the methods described herein include donepezil (Aricept ®), rivastigmine (Exelon®), memantine (Namenda®), and galantamine (Razadyne™ or Reminyl). In some embodiments, the composition also includes an inorganic nitrate; in other embodiments, the composition excludes inorganic nitrates. For example, the present composition can include inorganic nitrite and nitrates in a ratio that is between 1-5 to 1-100 nitrite:nitrate, e.g., 1-5, 1-10, 1-30, 1-50, 1-70, or 1-100 nitrite:nitrate.

### EXAMPLES

### Example 1

### Pharamceutical Nitrite Formulations

Pharmaceutical nitrite formulations can be prepared as described herein. Additional formulations are described in U.S. patent application Ser. No. 12/904,791, which is hereby incorporated by reference.

Controlled and Immediate Release Pharmaceutical Formulations

Exemplary formulations for oral administration include tablet and capsule formulations. For example, the powdered components described for a tablet formulation can be used to prepare a capsule formulation, a suitable capsule size depending on the dose of the active and density of the fill, such as size 1, 0, or 00 capsules. In some embodiments, the table or capsule may not have an enteric coating. In other embodiments, the pharmaceutical compositions of the invention can be formulated for controlled release of nitrite ion. If a capsule is described as coated, the coating can be applied to the capsule after filling. Capsule formulations can optionally employ self-locking capsule shells (e.g., Coni-Snap®, Posilok®, Snap-Fit®, or the like) for ease of handling during the coating process.

The exemplary compositions include between 0.5-4.0 mmol of total nitrite ion; specifically, between 1.8-3.6 mmol of NaNO₂. The compositions can include any prodrug of nitrite thereof, e.g., 125-250 mg of NaNO₂, 154-308 mg of KNO₂, or 201-402 mg of arginine nitrite. The amount of nitrite ion used in the pharmaceutical compositions can be varied as described herein. For example, the formulations can also include any of the excipients described herein, preferably an alkanizing agent (e.g., sodium bicarbonate or calcium carbonate), a glidant (e.g., fumed silica), a lubricant (a fatty acid salt (e.g., magnesium stearate), a pure solid fatty acid, or solid polyethylene glycol), or a bulking agent with good flow properties (e.g., silicified microcrystalline cellulose (Prosolv® SMCC90)). The compositions can also include any of the excipients described for use in compositions that are formulated for enteric release, e.g., in enteric formulations. Formulations can also include rate-controlling polymer coatings (e.g., ethyl cellulose, cellulose acetate, cellulose acetate butyrate, cellulose triacetate and the like, which can be combined with PEG-4000). If desired, the amount of PEG-4000 used can be varied in order to generate aqueous pores in the coat through which the sodium nitrite can diffuse. Enteric polymer coatings can also be used, and exemplary polymers include cellulose acetate phthalate (CAP), cellulose trimellitate, hydroxypropylmethylcellulose acetate succinate, Eudragit® L or S, or the like Where a polymer coating is used, the formulation can also include a plasticizer (e.g., triethylcitrate, triacetin, acetyl monoglycerides, or the like). The total enteric coat (polymer+plasticizer) can be added in an amount that, for example, results in a 10% weight gain.

In some embodiments, the pharmaceutical composition can be formulated as an enteric coated capsule.

The production and testing of several tablet and pellet formulations for the controlled release of nitrate is described in U.S. patent application Ser. No. 12/904,791, incorporated herein by reference.

Sodium Nitrite Release Assay

The UV absorbance at 355 nm can be measured with a Hewlett-Packard® 8453 diode-array UV-visible spectrophotometer for each release sample in a 10-cm quartz cuvette.

From a previously prepared calibration plot, the concentration of sodium nitrite in each sample can be calculated and converted to total amount and percent released for each tablet. The average percent released and standard deviation were calculated for two or three tablets run simultaneously. The average percent released vs. time profiles were plotted for each formulation.

Exemplary release profiles are described in described in U.S. patent application Ser. No. 12/904,791.

### Example 2

### Therapies for Age-Related Dementia

The therapeutic effects of nitrite treatment for age-related dementia can be studied clinically. For exemplary reviews, see, e.g.: Stein et al., “The Assessment of Changes in Cognitive Functioning: Reliable Change Indices for Neuropsychological Instruments in the Elderly—A Systematic Review,” Demnt. Geriatr. Cogn. Disord. 29:275-286 (2010); Frisoni et al., “The Clinical Use of Structural MRI in Alzheimer Disease,” Nat. Rev. Neurol. 6(2):67-77 (2010); Thompson et al., “Health- and Disease-Related Biomarkers in Aging Research,” Res. Gerontol. Nurs. 2(2):137-148 (2009); Vitali et al., “Neuroimaging in Dementia,” Semin. Neurol., 28(4):467-483 (2008); Salmon et al., “Neuropsychological Assessment of Dementia,” Annu. Rev. Psychol. 60:257-282 (2009); and Chao et al., “Cerebral Amyloid Angiopathy: CT and MR Imaging Findings,” RadioGraphics, 26:1517-1531 (2006). Still other references include: Jiwa et al, “Experimental Models of Vascular Dementia and Vascular Cognitive Impairment: A Systematic Review,” J. Neurochem. 115(4):814-828 (2010); Thal et al, “Cerebral Amyloid Angiopathy and its Relationship to Alzheimer's Disease,” Acta Neuropathol. 115(6):599-609 (2008); and Rhodin et al., “A Vascular Connection to Alzheimer's Disease,” Microcirculation, 8(4):207-20 (2001).

For example, elderly individuals with suspected dementia can be identified based on the presence of pharmacological dementia treatment (e.g., individuals who use any of the following medications: donepezil (Aricept®), rivastigmine) (Exelon°), memantine (Namenda®), and galantamine (Razadyne™ or Reminyl). Subjects on any of these medications or any combination thereof would be screened utilizing the Mini-Mental Status Examination to document the presence of cognitive dysfunction associated with dementia (MMSE total score <25). Subjects with an MMSE <25 would then undergo more extensive neuropsychological evaluation utilizing the Repeateable Battery for the Assessment of Neuropsychological Status (RBANS) to measure baseline performance in an array of cognitive domains. RBANS is a brief, individually administered test measuring attention, language, visuospatial/constructional abilities and immediate and delayed memory. It consists of 12 subtests which yield five Index scores and a Total score.

Subjects will also be administered the 18 point Clock Drawing Test (CDT) as a measure of global cognitive impairment. After documenting a cognitive profile consistent with dementia, the subjects would then undergo a clinical evaluation to ensure that the subject does not have confounding neurological disease (traumatic brain injury, Parkinson's disease, etc.) and to document medical history consistent with AD or vascular dementia (Hachinski Total Score of >4). Lastly, participants will be administered a Clinical Dementia Rating (CDR) test which is a subjective staging tool which has been used to document the progression of dementia.

Administration of the MMSE, RBANS, CDT, and CDR would be obtained on both groups at enrollment and six months to objectively document changes in cognitive function over that time frame.

Functional MRI (fMRI) can also be performed at enrollment and 6 month follow-up examinations.

In addition to clinical studies, animal models of CAA can be used. For example, a mouse model of CAA can be employed, which exhibits neuropathology identical to what is observed in human CAA, and also exhibits a concomitant cognitive decline. Amyloid deposition and cognitive decline can be measured in CAA mice using a large colony of mice of different ages. The mouse model develops initial deposition at 4-6 months of age, which becomes progressively worse over next 12 months and then plateaus. Cognitive decline is first observable at 8 months of age and becomes more severe over next 10 months. Sodium nitrite can be administered to mice beginning at 6 months of age (low dose, high dose, and vehicle) in the drinking water. Water intake would be monitored in order to obtain precise dosages, and the effects of 3 month and 6 month treatments on the primary endpoints of amyloid deposition and cognitive decline would be analyzed according to methods known in the art (e.g., Stone maze, fear conditioning, rotarod). Secondary endpoints will include measuring levels of nitric oxide synthase at sites of vascular amyloid deposition, collagen staining for vascular abnormalities, and inflammatory signaling (GFAP, IBA-1).

### Other Embodiments

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth.

All references, patents, patent application publications, and patent applications cited herein are hereby incorporated by reference to the same extent as if each of these references, patents, patent application publications, and patent applications were separately incorporated by reference herein.

What is claimed is:

1. A method for treating or preventing a condition that benefits from increasing cerebral blood flow in a mammal, or for increasing cerebral blood flow in a mammal diagnosed with a condition selected from cerebral amyloid angiopathy, age-associated dementia, and cognitive decline, wherein said method comprises the administration of an effective amount of inorganic nitrite, or a pharmaceutically acceptable salt, solvate, or prodrug thereof.

2. (canceled)

3. The method of claim 1, wherein said inorganic nitrite, or a pharmaceutically acceptable salt, solvate, or prodrug thereof, is administered as a pharmaceutical composition further comprising a pharmaceutically acceptable excipient.

4. The method of claim 1, wherein administration of said pharmaceutical composition to a human results in a peak plasma concentration of nitrite ion between 0.05 μM-10 μM.

5. The method of claim 4, wherein said peak plasma concentration of nitrite ion is between 0.05 μM-5 μM.

6. The method of claim 4, wherein said peak plasma concentration of nitrite ion is maintained for up to 14 hours.

7. The method of claim 6, wherein said peak plasma concentration of nitrite ion is maintained for up to 1 hour.

8. The method of claim 1, wherein said condition is dementia or cognitive decline.

9. The method of claim 8, wherein said dementia is age-associated dementia or vascular dementia.

10. The method of claim 8, wherein said dementia is mild to moderate dementia of the Alzheimer's type or dementia associated with Parkinson's disease.

11. The method of claim 8, wherein said dementia is moderate to severe dementia of the Alzheimer's type or dementia associated with Parkinson's disease.

12. The method of claim 1, wherein said condition is cerebral amyloid angiopathy.

13. The method of claim 1, wherein said mammal is a human.

14. The method of claim 13, wherein said inorganic nitrite is administered at a dose that is between 0.05 mg-5 mg/kg weight of the human.

15. The method of claim 3, wherein said pharmaceutical composition is formulated for oral administration.

16. The method of claim 3, wherein said pharmaceutical composition is formulated for extended release.

17. The method of claim 3, wherein said pharmaceutical composition is formulated for immediate release.

18. The method of claim 3, wherein said pharmaceutical composition is administered concurrently with one or more therapeutic agents for the treatment or prevention of a neurological disorder.

19. The method of claim 18, wherein said neurological order is a neurodegenerative disease.

20. The method of claim 18, wherein said therapeutic agent is donepezil, rivastigmine, memantine, or galantamine.
